(19) United States
(12) Patent Application Publication
(10) Pub. No.: US 2018/0228643 A1
(43) Pub. Date: Aug. 16, 2018

(54) POSITIONING MOUTHPIECE

(71) Applicant: Joseph Goldstein, Los Angeles, CA (US)

(72) Inventor: Joseph Goldstein, Los Angeles, CA (US)

(21) Appl. No.: 15/845,516

(22) Filed: Dec. 18, 2017

Related U.S. Application Data
(63) Continuation of application No. 14/475,104, filed on Sep. 2, 2014, now abandoned.
Application No. 14/475,104 is a continuation of application No. 14/254,203, filed on Apr. 16, 2014, now abandoned.
(60) Provisional application No. 61/855,283, filed on May 13, 2013.
(60) Provisional application No. 61/854,168, filed on Apr. 19, 2013.

(51) Int. Cl. A61F 5/56; A61C 5/90; A61M 16/04

(52) U.S. Cl. CPC A61F 5/566; A61C 5/90; A61M 2205/0216; A61M 2210/0637; A61M 16/049

(57) ABSTRACT

A positioning mouthpiece comprises an upper component having a mouth portion and a fastening portion, and a lower component having a mouth portion and a fastening portion. The fastening portion of the upper component and the fastening portion of the lower component are attached to each other such that limited back and forth movement and lateral movement of the mouth portion of the upper component and the mouth portion of the lower component can take place.

### CROSS-REFERENCE TO RELATED APPLICATIONS

This application is a continuation-in-part of U.S. patent application Ser. No. 14/254,203 filed Apr. 16, 2014, which claims the benefit of Provisional Patent Application No. 61/854,168 filed Apr. 19, 2013, and U.S. Provisional Patent Application No. 61/855,283 filed May 13, 2013, both of which are incorporated herein by reference in their entirety.

### FIELD AND BACKGROUND OF THE INVENTION

This invention relates to a positioning mouthpiece. Particularly, the positioning mouthpiece of the invention is one which may be used so as to position the tongue and the palate relative to each other, and may be worn beneficially by, for example, sufferers from sleep apnea, usually as an accessory or in combination with other equipment. The positioning mouthpiece may be configured so as to permit at least some forward and backward movement as well as lateral movement of the lower jaw relative to the upper jaw.

The positioning mouthpiece of the invention may have multiple applications other than for use in treating sleep apnea, however, and can be used to advantage in a plurality of contexts to aid in the comfort and/or treatment of the user.

### SUMMARY OF THE INVENTION

According to one aspect of the invention, there is provided a positioning mouthpiece comprising: an upper component having a mouth portion and a fastening portion; a lower component having a mouth portion and a fastening portion; and wherein the fastening portion of the upper component and the fastening portion of the lower component attached to each other such that limited back and forth movement and lateral movement of the mouth portion of the upper component and the mouth portion of the lower component can take place.

In one embodiment, the mouth portion of the upper component comprises a base wall and two sidewalls defining a channel for receiving the upper arch of the user. Further, a material may be located within the channel, the material being configured to the shape of the user's dentition. Preferably, a slot is formed in at least one of the sidewalls defining the channels, the slot being positioned to receive and hold the material located within the channel.

In one form of the invention, the base wall of the upper component has an inner surface and an outer surface, the outer surface having a plurality of protrusions thereon to slightly space the upper component from the lower component.

Preferably, the fastening portion of the upper component comprises a flat plate extending therefrom, the flat plates having connector means thereon for receiving the fastening portion of the lower component. The connector means may comprise a pair of opposing hook members extending downwardly from the fastening means, each hook member comprising a substantially vertical wall and a substantially horizontal wall at the end of the vertical wall, the hook members defining a space comprising the distance between the vertical walls and the height between the flat plate and the horizontal wall.

In one embodiment, the space has tolerances which allow limited movement of the fastening means of the lower component within the space when in the operable position to facilitate relative backward and forward movement and lateral movement between the upper component and the lower component.

In accordance with the invention, the mouth portion of the lower component may comprise a base wall and two sidewalls defining a channel for receiving the lower arch of the user. Further, a material may be located within the channel, the material being configured to the shape of the user's dentition.

Preferably, a slot is formed in at least one of the sidewalls defining the channels, the slot being positioned to receive and hold the material located within the channel.

In one embodiment, the fastening portion of the lower component comprises a plate extending therefrom. The plate of the lower component may have a wider portion attached to the mouthpiece portion, and a narrower portion. A step with an abutment surface may be formed at the juncture of the wider portion and the narrower portion.

Preferably, the fastening portion of the upper component comprises a flat plate extending therefrom, the flat plates having connector means thereon for receiving the fastening portion of the lower component, the connector means comprising a pair of opposing hook members extending downwardly from the fastening means, each hook member comprising a substantially vertical wall and a substantially horizontal wall at the end of the vertical wall, the hook members defining a space comprising the distance between the vertical walls and the height between the flat plate and the horizontal wall, and wherein the narrower portion of the plate of the lower component is received between the hook members.

In one embodiment, the space between the opposing hook members has tolerances which allow limited movement of the narrower portion of the fastening means of the lower component within the space when in the operable position to facilitate relative backward and forward movement and lateral movement between the upper component and the lower component.

The positioning mouthpiece may be connectable to further equipment for effecting treatments or procedures to the user.

According to a further aspect of the invention, there is provided a positioning mouthpiece comprising: an upper component having a mouth portion and a fastening portion; a lower component having a mouth portion and a fastening portion; wherein the fastening portion of the upper component and the fastening portion of the lower component are attached to each other so as to permit limited back and forth movement, as well as lateral movement, of the mouth portion of the upper component relative to the mouth portion of the lower component.

According to yet a further aspect of the invention, there is provided a method of positioning facial structures during a treatment or procedure, the method comprising: placing a mouthpiece of an upper component in the mouth of a user with a fastener extending outside the mouth; placing a mouthpiece of a lower component in the mouth of a user with a fastener extending outside the mouth; and connecting the fastener of the upper component with the fastener of the lower component in a nonrigid, flexible manner such that limited back and forth movement between the mouthpiece on the upper portion and the mouthpiece on the lower portion is facilitated. Limited lateral movement between the mouthpiece on the upper portion and the mouthpiece on the lower portion may also be facilitated.

This invention therefore relates in one aspect to a unique design for a dental mandibular advancement device for the treatment of respiratory diseases, for example, snoring and sleep apnea.

The device in one aspect consists of an upper portion which fixes to the patient's upper arch and a lower portion which affixes to patients or lower arch. The upper arch bracket is slotted and affixed to the lower portion by means of a fastening device so that the lower arch can be advanced forward or backward, or even laterally, so as to control the upper airway of the patient and the relative positioning of the jaw and mouth parts.

Since lateral right to left motion may provide a greater degree of patient comfort, the upper member may have a narrow bar or flat piece or other structure which fits into any of the concave, indented or other types of slots or structures of the lower member. The upper member may be reduced in size when compared to the lower slots or structure so that when the patient moves the chin or lower jaw, lateral facial motion may be possible, as well as forward and backward movement. This flexibility allows for a less intrusive device and less rigidity which can thereby reduce any pain or discomfort which may result from a rigid structure.

The positioning mouthpiece may be used in conjunction with a dental mandibular advancement device which may also have an extension member so that an air delivery device such as CPAP-BI-PAP, etc. interface can be utilized in conjunction with the positioning mouthpiece of the invention. While the use of the positioning mouthpiece with sleep apnea treatment devices is an important feature and aspect of the present invention, it should be noted that the invention is not so limited and may be used in many other contexts as well. It is also, therefore, a device that can be used generally in dental or mandibular treatments and therapies where it is desirable or necessary to relatively position the mouth, jaws and other facial structures in a manner which enhances or enables procedures which require for their effectiveness a reduced amount of relative movement between such structures.

For some respiratory ailments, such as sleep apnea, as an example, machines such as CPAP, BIPAP, and VIPAP are utilized to introduce pressurized air into the patient's body through the nostrils. Since many patients have a tendency to open their mouth during sleep when they suffer from sleep apnea, the regimen can easily become ineffective since the pressurized air is “short-circuited” out through the mouth.

At this time, two techniques may be utilized to circumvent or reduce this problem. The simplest, but generally more ineffective, is the use of a chin strap which is a resilient band that usually goes from the top of the patient's head to under the chin in an attempt to keep the mouth closed, but often fails to achieve this objective.

A more cumbersome and even more uncomfortable technique is the utilization of a “full face mask” which covers both the nostrils and the patient's mouth. Such a face mask may itself be hooked up to a device for delivering air at selected intervals, making it even more difficult to be attached to all of this equipment while trying to sleep. This technique may be extremely severe and difficult for a patient to sustain and, therefore, can often lead to patient noncompliance.

The positioning mouthpiece of the present invention, when used as a dental device for holding facial structures in preferred relative positions, may be configured to keep the mouth sufficiently closed to prevent the pressurized air from flowing out of the patient's mouth under normal operating conditions. It can be joined to many types of nasal interface devices such as CPAP masks and nasal puffs, as examples, that introduce pressurized air into the patient's nostrils. The positioning mouthpiece of the invention may be structured so as to provide a small airway to the outside of the mouth as safety precaution in case the attached sleep apnea devices fail to function partially or fully and oxygen supply may as a result be severed of diminished. The airway may be a gap or pathway between components of the positioning mouthpiece, dimensioned to allow air intake in case of such failure but otherwise not significantly affecting the operation of the sleep apnea equipment.

In accordance with one embodiment of the present invention, the positioning mouthpiece may comprise a two-piece joined combination dental appliance having two segments, part of which is in the mouth and a part of which is outside the mouth. One segment may affix to the upper arch or teeth in a comfortable secure manner; the other segment may be affixed in a comfortable secure manner to the patient's lower arch or teeth. The locking force of the upper and lower components or segments is sufficient to keep the patient's mouth from opening to an extent which permits the output flow of pressurized air through the mouth of the user.

Through the utilization of a connector type member that joins the upper and lower components or segments, back and forward motion is to some extent permitted while the device is in the mouth and in the operating condition. Further, lateral facial motion, such as of the lower jaw relative to the upper jaw, is facilitated providing additional patient comfort. The comfort features built in to the positioning mouthpiece of the invention are beneficial on many levels, and also help ensure that the person will continue to use the device, as opposed to discontinuing the use because of the rigidity and discomfort which occur in other mouthpieces.

In one embodiment, the positioning mouthpiece may be used in conjunction with an extension bracket or other component which extend outwardly between the lips, and the appliance can be affixed to nasal interface devices. While this is one use of the invention, it is not the only context in which the positioning mouthpiece may be utilized. For example, the positioning device may be used in conjunction with dental or facial treatments and procedures where it is desirable or necessary to keep the movement of the mouth limited or restricted to reap the optimal benefits of such treatments.

With a positioning mouthpiece of the invention, the need for a chin strap, a full face mask, or such other cumbersome and invasive devices can be effectively reduced or even eliminated, thereby improving patient comfort and compliance.

### BRIEF DESCRIPTION OF THE DRAWINGS

In the drawings:

FIG. 1 is a top perspective view of a positioning mouthpiece in accordance with one aspect of the invention, showing both the upper and lower components thereof connected to each other;

FIG. 2 is a bottom perspective view of the positioning mouthpiece as shown in FIG. 1 of the drawings;

FIG. 3 is a top end perspective view of the positioning mouthpiece as shown in FIG. 1 of the drawings;

FIG. 4 is a is a bottom end perspective view of the positioning mouthpiece as shown in FIG. 1 of the drawings;

FIG. 5 is an end view of the positioning mouthpiece as shown in FIG. 1 of the drawings;

FIG. 6 is a side view of the positioning mouthpiece as shown in FIG. 1 of the drawings;

FIG. 7 is a further side view of the positioning mouthpiece as shown in FIG. 1 of the drawings;

FIG. 8 is a top perspective view of the top component of the positioning mouthpiece in accordance with an aspect of the invention;

FIG. 9 is a bottom perspective view of the top component of the positioning mouthpiece as shown in FIG. 8 of the drawings;

FIG. 10 is a top view of the top component of the positioning mouthpiece as shown in FIG. 8 of the drawings;

FIG. 11 is a bottom view of the top component of the positioning mouthpiece as shown in FIG. 8 of the drawings;

FIG. 12 is a side view of the top component of the positioning mouthpiece as shown in FIG. 8 of the drawings;

FIG. 13 is a side perspective sectional view of the top component of the positioning mouthpiece as shown in FIG. 8 of the drawings;

FIG. 14 is a detail side perspective view of the top component of the positioning mouthpiece as shown in FIG. 8 of the drawings, showing part of the fastening means;

FIG. 15 is an end view of the top component of the positioning mouthpiece as shown in FIG. 8 of the drawings;

FIG. 16 is a top perspective view of the bottom component of the positioning mouthpiece in accordance with an aspect of the invention;

FIG. 17 is a bottom perspective view of the bottom component of the positioning mouthpiece as shown in FIG. 16 of the drawings;

FIG. 18 is a top view of the bottom component of the positioning mouthpiece as shown in FIG. 16 of the drawings;

FIG. 19 is a bottom view of the bottom component of the positioning mouthpiece as shown in FIG. 16 of the drawings;

FIG. 20 is a side view of the bottom component of the positioning mouthpiece as shown in FIG. 16 of the drawings;

FIG. 21 is a side perspective sectional view of the bottom component of the positioning mouthpiece as shown in FIG. 16 of the drawings;

FIG. 22 is a detail side perspective view of the bottom component of the positioning mouthpiece as shown in FIG. 16 of the drawings, showing part of the channel for receiving the teeth or jaw of the user;

FIG. 23 is an end view of the bottom component of the positioning mouthpiece as shown in FIG. 16 of the drawings; and

FIG. 24 is a schematic view of the positioning mouthpiece located in the mouth of a user so as to relatively position the tongue and palate.

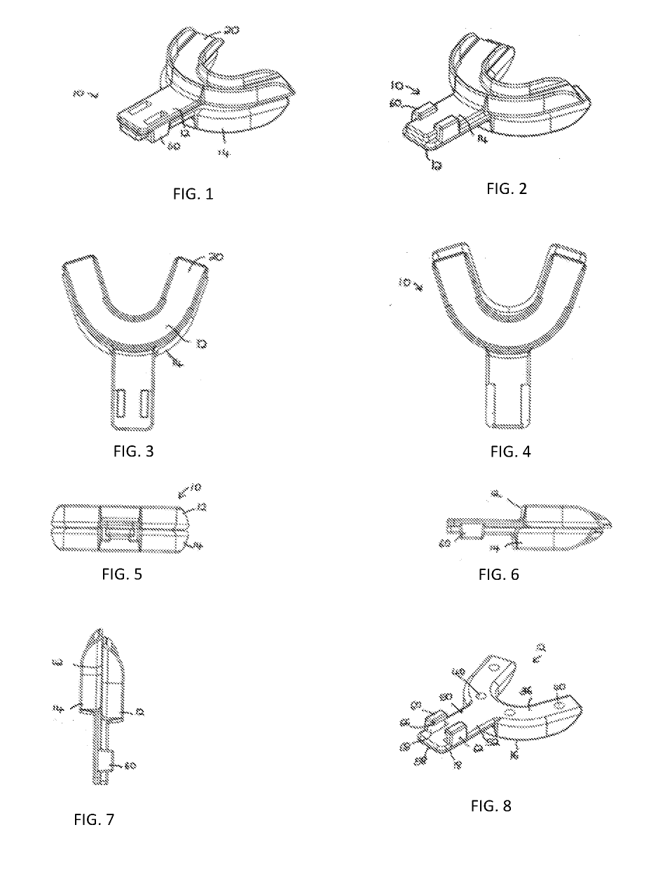

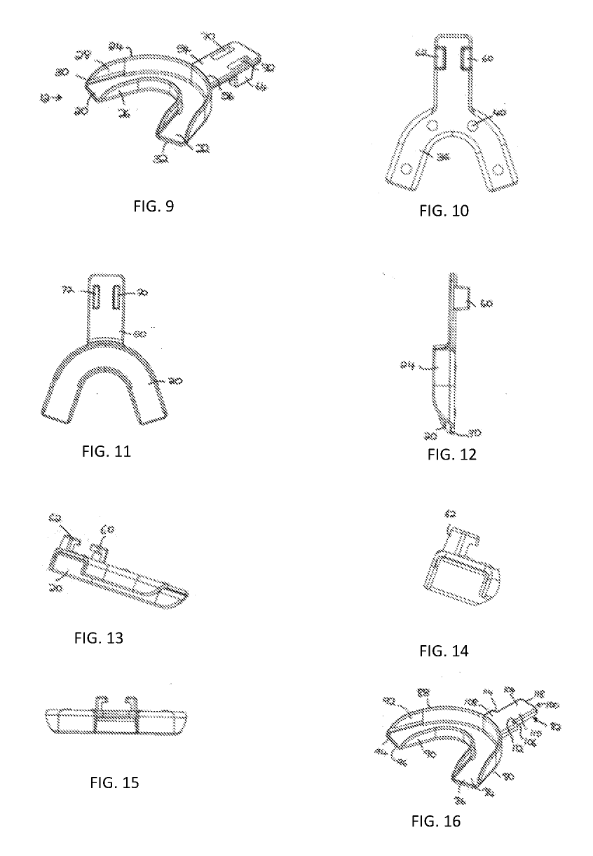

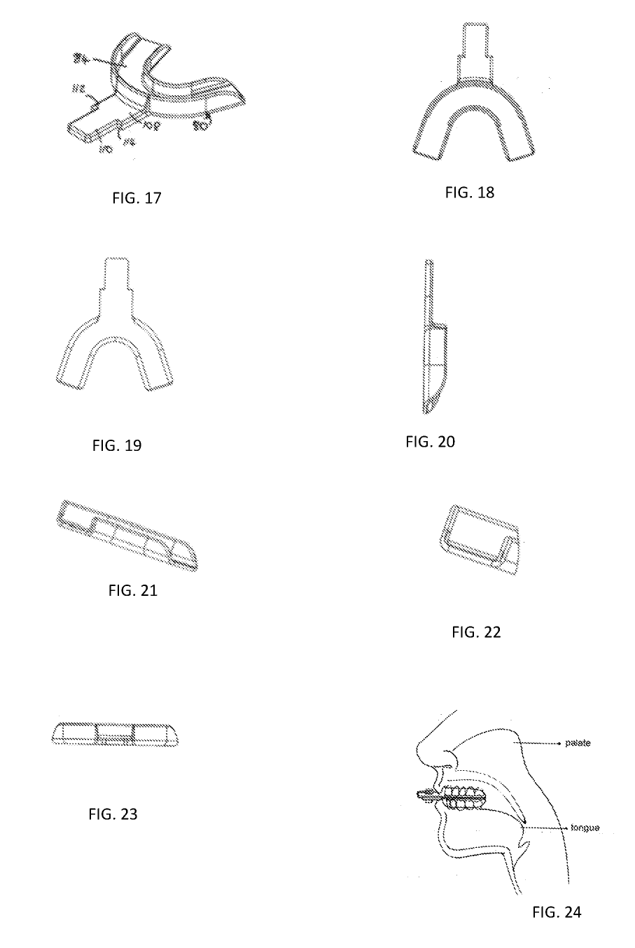

### DETAILED DESCRIPTION OF THE INVENTION

Reference is now made to the drawings which show various embodiments and views of the positioning mouthpiece in accordance with aspects of the invention. The drawings are intended to be exemplary only, and do not define the limits or scope of the invention. Variations of the positioning mouthpiece are possible within the scope of the invention.

As mentioned, the positioning mouthpiece of the invention may have particular application in the area of sleep apnea treatment, providing a mouthpiece which assists the user in keeping the mouth positioned for optimal use of provided oxygen, in a more comfortable and user friendly manner. However, the positioning mouthpiece of the invention can be used in a variety of other contexts as well, whenever carrying out dental or facial procedures demands or benefits from the presence of a device which is able to restrict in some manner the movements of the jaw or other facial structures. In such cases, the positioning mouthpiece of the invention provides a good solution with a more user friendly flexible device which addresses not only the proper positioning of the facial structures but also the comfort and convenience of the user.

Referring to the drawings, there is shown a positioning mouthpiece device 10 which has an upper component 12 and a lower component 14. The upper component 12 and lower component 14 can be connected or fastened to each other in the manner illustrated in the drawings, and, as will be described, such that the upper component 12 is engaged by the upper jaw or teeth or arch of the user, while the lower component 14 is engaged by the lower jaw or teeth or arch of the user. Relative movement within defined parameters is possible between the upper component 12 and lower component 14 to allow some additional flexibility and comfort but without compromising the purpose of the positioning mouthpiece device 10, namely, to keep at least some of the various facial structures to which the positioning mouthpiece device 10 directly or indirectly attaches from moving relative to each other beyond the selected amounts or tolerances.

The upper component 12 comprises a mouth engaging portion 16, for releasable attachment within the mouth of the user, and a connecting portion 18 by means of which the upper component 12 can be connected to the lower component 14, as will be described in further detail below. The mouth engaging portion 16 essentially comprises a channel 20 formed by a base wall 22, an outer side wall 24, and in inner side wall 26. The channel 20 has an open top 28, through which a wax or other substance can be poured and which can assume the shape of the jaw and teeth, and also through which the upper teeth of the user can pass so that the upper component 12 can engage with the teeth. It will be noted that the side walls 24 and 26 both have recessed slots 30 near the rear end 32 of the channel 20, and these slots 30 help to keep the wax or other substance, when hardened or solidified, locked in position to prevent it from falling out of the channel 20. The wax will typically be poured into channel 20, including the slots 30, in a liquid or viscous state, and thereafter harden and set therein. After hardening, the wax will be captured and lodge within the slots 30, and this will therefore reduce the possibility of the set wax from falling out of the channel 20 after hardening.

The base wall 22 has an outer surface 36 which in use abuts up against a corresponding surface of the lower component 14. The outer surface 36 has formed thereon a series of small protrusions 40 extending outwardly therefrom. The function of these protrusions 40 is to ensure that in use a space or gap 42 is formed between the upper component 12 and the lower component 14 (best seen, for example, in FIGS. 5, 6 and 7 of the drawings) so that an airway or air passage is created. Such an air passage will ensure that the user's mouth still has access to outside ambient air when the positioning mouthpiece device 10 is located in the mouth. This may be important if, for example, the sleep apnea equipment to which the positioning mouthpiece 10 may be connected for the supply of air malfunctions or the air supply is interrupted. In such an event, the user will still be able to breath in air from the outside through the gap 42 created by the protrusions 40. The construction to create the gap 42 between the upper 12 and lower 14 components therefore provides a useful safety feature in these circumstances, ensuring the access to ambient air. However, the gap 42 is constructed so as to have dimensions which do not otherwise interfere with the effective operation of the sleep apnea or other equipment which may be used in conjunction therewith.

The connector portion 18 of the upper component 12 is a tongue-like structure which is comprised of a flat plate 50 having an upper surface 52 and a lower surface 54. The edges of the flat plate 50 may be beveled for easier handling. The flat plate 50 has a connected end 56 which attaches to outer side wall 24 of the channel 20 at about its midpoint and a remote or free end 58 at its opposite end. Extending downwardly from the upper surface 52 of the flat plate 50, near the free end 58 thereof, are a pair of hook connectors 60 and 62. Each hook connector 60 and 62 comprises a connector wall 64 and a flange 66 extending inwardly from the end of the connector wall 64. The connectors 60 and 62 together define a space 68 between them which receives the lower component 14, as will be described. The space 68 is bounded by the inner surfaces of the connector walls 64 and the height between the inner surface of the flange 66 and the upper surface 52.

A pair of rectangular apertures 70 and 72 are formed in the flat plate 50, each of the apertures 70 and 72 being at the region of the connectors 60 and 62 and preferably located below the flanges 66 thereof.

The structure of the lower component 14 will now be described. The lower component 14 comprises a mouth engaging portion 80 and a connector portion 82.

The mouth engaging portion 80 essentially comprises a channel 84 formed by a base wall 86, an outer side wall 88, and in inner side wall 90. The channel 84 has an open top 92, through which a wax or other substance can be poured, and also through which the lower teeth of the user can pass so that the lower component 14 can engage with the teeth. It will be noted that the side walls 88 and 90 both have recessed slots 94 near the rear end 96 of the channel 84, and these slots 94 help to keep the wax or other substance in position to prevent it from falling out of the channel 84, in substantially the same manner as has already been described with reference to the upper component 12 above.

The base wall 86 has an outer surface 98 which in use abuts up against the corresponding outer surface 36 of the upper component 12. The outer surface 98 of the lower surface is kept spaced from the outer surface 36 of the upper component 12 by the series of small protrusions 40 extending outwardly therefrom, as described above. The protrusions 40 ensure that the space or gap 42 is formed between the upper component 12 and the lower component 14 (best seen, for example, in FIGS. 5, 6 and 7 of the drawings) so that an airway or air passage is created, as has already been described above.

The mouth engaging portion 80 of the lower component 14 has the connector portion 82 extending outwardly therefrom. In use, the mouth engaging portion 80 will be received in the mouth of the user and clamped between the upper and lower jaws or teeth, and the connector portion 82 will be external to the mouth and connect to the connector portion 18 of the upper component 12, as will be described.

The connector portion 82 comprises a plate 100 which extends away from the outer side wall 88 from approximately the middle or center thereof. The plate 100 has an upper surface 102 and a lower surface 104, as well as the edges 106 which provide thickness to the plate 100. Further, the plate has a wider portion 108 and a narrower portion 110 with a step 112 between them. The step 112 has an abutment surface 114 which engages with the connectors 60 and 62 of the upper component 12 which thereby limits the extent of relative forward and backward movement between the upper component 12 and the lower component 14. In this way, the lower component 14 will only be able to move up to this defined position relative to the upper component 12 in order to achieve the objective of the positioning mouthpiece 10 in keeping the specific facial structures from moving relative to each other beyond a certain amount.

The operation of the positioning mouthpiece device 10 and interrelationship between the upper component 12 and the lower component 14 will now be described. The upper component 12 and the lower component 14 can be releasably affixed to each other in a way which ensures that there is still movement allowable between the upper and lower components 12 and 14, but not enough movement to defeat the purpose of the treatment or procedure with which the positioning mouthpiece device 10 is associated. The plate 100 of the connector portion 82 on the lower component 14 is placed over and adjacent the flat plate 50 of the connector portion 18 on the upper component 12. The outer surface 36 of the upper component 12 will be right near the outer surface 98 of the lower component 14.

The leading edge 118 of the plate 100 is advanced over the flat plate 50 until it passes under the flange 66 of the connectors 60 and 62. Once captured under the connectors 60 and 62, the plate 100 will be held in position and the upper component 12 becomes attached to the lower component 14, in the sense that they can no longer be pulled apart. But relative movement between the upper component 12 and the lower component 14 will still be possible. First, relative sliding movement between the two can be achieved by moving the plate 100 over the flat plate 50. Eventually, the abutment surface 114 of the step 112 will engage the connector wall 64, and further movement will be prevented. This therefore serves as the limit to which the lower component 14 can slide over the upper component 12. It should be noted that, at least in one embodiment, the thickness of the plate 100 is slightly less than the height of the space 68 (seen in FIG. 5 of the drawings), this height being defined as being the distance between the upper surface 52 of the flat plate 50 and the lower or under surface of the flange 66.

Furthermore, at least in one embodiment, the width of the plate 100 at the narrower portion 110 thereof may be slightly less than the distance between the inside surface of the connector walls 64, as defined by the space 68. These tolerances allow the lower component 14 to move to some extent relative to the upper component 12. Thus, the upper component is allowed limited movement in the backward and forward directions as it slides axially under the connectors 60 and 62. Also, the upper component 12 can move slightly laterally relative to the lower component 14 because the width of the narrower portion 110 of the plate 100 is just a little less than the width of the space 68. The net effect of these permitted movements is to allow the user some flexibility and the discomfort and even pain associated with a rigidness of other types of mouthpieces can reduced or eliminated. At the same time, the structure of the positioning mouthpiece device 10 nevertheless continues to hold the facial structures in their preferred positions within the acceptable limits as may be determined by the treatments and procedures being applied so that the purpose of the device is not compromised.

In use, the channels 20 and 84 are filled with a wax or other substance which will take the shape and configuration of the dentition or arch of the user. One such wax or substance that may be used is Elvax 150™ made by Dupont.

FIG. 24 of the drawings shows a schematic representation of a positioning mouthpiece device 10 in accordance with the invention located in the mouth of a user. The device 10 keeps the palate and tongue properly positioned relative to each other to facilitate treatments and procedures. One such treatment involves the use of sleep apnea or equipment. The positioning mouthpiece device 10 of the invention allows such equipment to operate more effectively by maintaining the mouth structures in the correct position. Moreover, it provides a small gap or airway in case of equipment failure or interruption, as a safety precaution.

When the positioning mouthpiece device 10 of the invention is affixed to the user's or patient's teeth, the mouth opening is sufficient for oral breathing, but minimized to the extent that the proper tongue and palate physiological closure relationship is effective for the encouragement of nasal breathing.

The positioning mouthpiece device 10 may be comprised of FDA approved biocompatible Elvax™ for traditional “boil and bite” creation of the dental impression of the upper and lower arch. This material would be placed in the various channels of the positioning mouthpiece device 10 and the impression therein is of course specific to the user's dentition. The positioning mouthpiece device 10 may itself be comprised of an FDA approved biocompatible polycarbonate, although the invention is not limited to such a material. Any other suitable material, or a combination of materials, can be used to construct the positioning mouthpiece 10 of the invention.

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

1. An improved method of treating sleep apnea in a user by delivering pressurized air from a sleep apnea treatment device, the improvement being reducing the amount of pressurized air lost through the user's mouth during sleep by insertion of a positioning mouthpiece in the user's mouth, the positioning mouthpiece being comprised of:
(i) an upper component configured to affix to the user's upper dental arch or upper teeth in a comfortable secure manner, and a lower component configured to affix to the user's lower dental arch or lower teeth in a comfortable secure manner, wherein
(ii) each of the upper component and lower component has a fastening portion, and the fastening portion of the upper component and the fastening portion of the lower component attach to each other so as to
(a) permit forward-and-backward movement as well as lateral movement of the user's lower jaw relative to the user's upper jaw whole and
(b) create a locking force sufficient to keep the user's mouth from opening to an extent that pressurized air is short circuited and flows out through the user's mouth.

2. The positioning mouthpiece of claim 1 wherein the upper component is comprised of a base wall and two sidewalls defining a channel shaped for receiving the upper dental arch or upper teeth of the user and the lower component is comprised of a base wall and two sidewalls defining a channel shaped for receiving the lower dental arch or lower teeth of the user.

3. The positioning mouthpiece of claim 2 wherein the base wall of the upper component has an inner surface and an outer surface, and the outer surface has a plurality of protrusions thereon to slightly space the upper component from the lower component.

4. The positioning mouthpiece of claim 2 having connector type member that joins the upper and lower components.

5. The positioning mouthpiece of claim 2 wherein the fastening portion of the upper component comprises a flat plate extending therefrom, and the flat plate has a connector means thereon for receiving the fastening portion of the lower component.
